(19) United States
(12) Plant Patent Application Publication
(10) Pub. No.: US 2005/0132457 P1
(43) Pub. Date: Jun. 16, 2005

(54) Geranium plant named Rebekah

(50) PELARGONIUM HORTORUM; GERANIUM

(76) Inventor: Laura B McCarth Pres Bartlett, Sudbury, MA (US)

Correspondence Address: J P BARTLETT CO INC, 578 BOSTON POST RD, SUDBURY, MA, 01776, US

(21) Appl. No.: 10/736,201

(22) Filed: Dec. 15, 2003

(51) Int. Cl. A01H 5/00

(52) U.S. Cl. PLT/329

(57) ABSTRACT

This invention is a new and distinct cultivar of a zonal geranium (Pelagronium×hortorum) named ‘Rebekah’ the cultivar has a brilliant array of colors within the plant's purple blossom and medium green foliage on a stable, full, well branched plant.

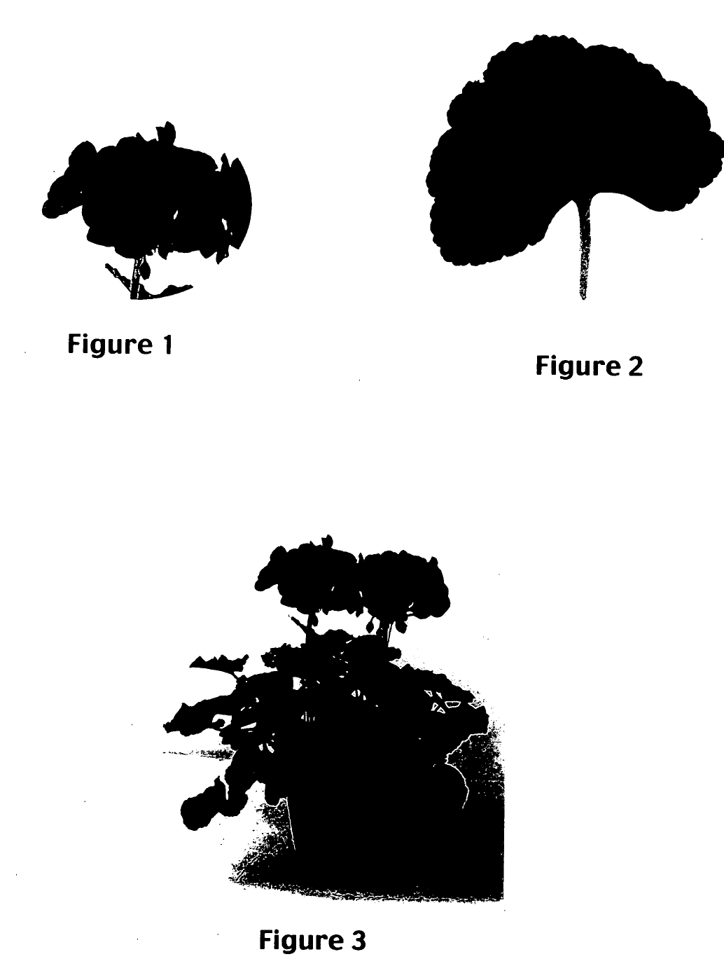

### BOTANICAL CLASSIFICATION/CULTIVAR DESIGNATION

Pelargonium.times.hortorum cultivar ‘Rebekah’

### BACKGROUND OF THE INVENTION

The cultivar was developed from a controlled breeding program conducted at the J.P. Bartlett Company. The company used a non-patented cultivar designated #94362 as the male parent and a non-patented cultivar designated #2484 as the female parent in a cultivated area to create ‘Rebekah’. ‘Rebekah's’ plant habit is symmetrical with excellent basal as well as a lateral branching habit. Asexual propagation of the new cultivar by cuttings at the location previously stated has shown that the unique features of this new geranium are stable and that the plant reproduces true to type in successive generations of asexual propagation.

### SUMMARY OF THE INVENTION

The plants growth habit is compact to medium in vigor. It is a full plant that performs well in both a greenhouse and in a natural environment such as a garden. It will grow more compact under drier conditions but has the ability to be a larger and more vigorous plant with sufficient water and partial shade. The blossoms are tolerant to rain, not turning brown and rotting after a day or so of straight rain. Rebekah buds early and is consistent with a large and long lasting blossom than many other cultivars known to me. On average, the plant itself can get to over 324 millimeters tall, not including the stem or flower. The free branching nature of Rebekah allows the plants girth to grow and flourish. The width of a single plant is measured to be 563 millimeters and growing. The vibrancy of blossom color and strong green leaf color separates itself from any other known to me. The semi-double blossom is a dark purple/violet with a darker edge to each petal. ‘Rebekah's’ stem is a distinguished light green, while the leaf produces a unique variation of colors all in its own. ‘Rebekah's’ standout performance is regarded highly due to it's excellent growth habit, size, blossom and leaf. The new cultivar of the present invention is being marketed under the BARTLETT'S trademark.

### BRIEF DESCRIPTION OF THE PHOTOGRAPH

Accompanying this application is a photographed picture of the plant and its' aspects.

1. Figure one shows the large, semi-double blossom and the purple/violet color described in the summary.

2. Figure two enables us to see the medium green leaf with the medium green zone.

3. Figure three shows the overall plant. This is a single plant from a cutting in a four-inch pot. The height, width, and growth habit are clearly seen here.

### DETAILED BOTANICAL DESCRIPTION

The following observations and measurements describe asexual reproductions of ‘Rebekah’ grown in greenhouses located in Sudbury, Mass., USA. The plant history was taken on 3 month old plants grown in 7.5″ pots, blossomed under natural light in a greenhouse and color readings were taken in the greenhouse in Sudbury, Mass. under ambient light. Color references are primarily to the Pantone Color Selector Chart 1000/coated 1995. The color values were determined on Oct. 22, 2003 under natural light conditions of 200 foot-candles.
Classification:
   Botanical.—Pelargonium×hortorum Bailey, cv. ‘Rebekah’.
Inflorescence:
A. Umbel:
   Average diameter.—Approximately 8.5 to 10.5 cm.
   Average depth.—Approximately 4.75 to 6.5 cm.
   Peduncle length.—Approximately 14 to 16 cm.
   Pedicel length.—Approximately 2.5 to 3 cm.
   Number of umbels per plant.—When grown in a 7.5″ pot at 8 weeks after the sticking of a rooted cutting, there commonly are approximately 6 to 8 umbels per plant.
   Number of florets/umbel.—When grown in a 7.5″ pot at 8 weeks, approximately 24 to 28 florets per umbel commonly are formed.
B. Corolla:
   Buds.—Elliptical to round in configuration, initially light green and changing to dark violet at the time of opening, and approximately 12 to 15 mm. in length and approximately 6 to 8 mm. in width.
   Pedicels.—Commonly approximately 4 to 5.5 cm in length and curved which commonly causes the buds to point substantially downward.
   Average diameter.—Approximately 5 to 7 cm.
   Form.—Commonly semi-double with at least two petaloids.
   Petals.—Spatulate shaped, satiny and smooth. Commonly approximately 6 to 8 petals are present per floret.
   Number of petaloids.—Commonly forms approximately 2 to 5 petaloids per floret.
   Color.—General tonality from a distance of three meters: Dark Violet.
   Color of upper petals.—Pantone 233C.
   Markings of upper petals.—Orange spot at base of petal Pantone 1787C.
C. Reproductive organs:
   Androecium.—5 fertile anthers, with whitish filaments, orange pollen the lower side of the mature (open) anther is black.
   Gynoecium.—5 lobed violet stigma, Pantone 233C, greenish style; one pistil per flower.
   Fertility/seed set.—Commonly does not produce fruits in the absence of mechanical fertilization.
   Spring flowering response period.—Approximately 5 to 8 weeks from rooted cuttings under standard greenhouse conditions.
   Outdoor flower production.—Freely flowering under outdoor growing conditions with substantially continuous blooming.
   Durability.—Ships well.
   Pest/disease resistance/susceptibility.—No susceptibility to pests or diseases noted to date.
Plant:
   A. Foliage.—Medium green with slight zonation. The leaves are pubescent on the upper surface.
   Length.—3 to 4 cm.
   Width.—5 to 6 cm.
   Form.—Reniform, with a cordate base.
   Margin.—Irregularly crenate.
   Ribs and veins.—Palmate 5 to 6 cm. long.
   Color.—Upper side is Pantone 368C. Under side is Pantone 370C.
   Petioles.—Commonly approximately 3 to 6 cm.
A. General appearance and form:
   Internode length.—Commonly varies from approximately 0.6 to 0.9 cm.
   Branching pattern.—Freely basal branching as well as lateral branching. No pinching is required to obtain self-branching. A self-branching growth habit is observed in the absence of a growth regulator.
   Height.—Approximately 20 to 26 cm. above a 7.5″ pot at 8 weeks under standard greenhouse conditions.

1. J.P. Bartlett Co., Inc has bred a new and distinct geranium plant as shown and described
